(19) United States
(12) Patent Application Publication
(10) Pub. No.: US 2022/0280503 A1
(43) Pub. Date: Sep. 8, 2022

(54) COMPOSITIONS OF OPIOID ANTAGONISTS, IMPLANT DEVICES, AND TREATMENT METHODS FOR OPIOID USE DISORDER

(71) Applicant: Delpor, Inc., Brisbane, CA (US)

(72) Inventor: Gregory A. Watkins, Oakland, CA (US)

(21) Appl. No.: 17/637,756

(22) PCT Filed: Aug. 27, 2020
(86) PCT No.: PCT/US2020/048289

§ 371 Date: Feb. 23, 2022

Related U.S. Application Data
(60) Provisional application No. 62/893,097, filed on Aug. 28, 2019.

(51) Int. Cl. A61K 31/485; A61K 47/12; A61K 47/02; A61K 47/18; A61K 9/00; A61K 9/16; A61K 9/20

(52) U.S. Cl. CPC A61K 31/485; A61K 47/12; A61K 47/02; A61K 47/18; A61K 9/0024; A61K 9/1617; A61K 9/2013

(57) ABSTRACT

Compositions comprising a formulation of an opioid antagonist and an organic acid are described. The opioid antagonist is a base and has a water solubility at room temperature of less than about 1.0 g/L. The organic acid is combined with the opioid antagonist in a mole ratio less than or equal to 1:1 (acid:drug), has a water solubility at room temperature of between 0.1 and 10 g/L, and/or has a molar mass of less than 400 grams per mole. The organic acid enhances the solubility of the opioid antagonist, and thus facilitates the release of the opioid antagonist into a buffered environment of use for prolonged periods of, for example, six months to one year. Diffusion-based drug delivery devices comprising the compositions and methods of treatment are also described.

### TECHNICAL FIELD

The subject matter described herein relates to compositions for small molecule therapeutic agents to treat opioid use disorder (OUD), and to drug delivery devices that incorporate these compositions for the controlled, sustained delivery of small molecule opioid antagonists to treat OUD.

### BACKGROUND

Opioids are a class of drugs that includes both the illicit drug heroin and prescription pain relievers such as morphine, fentanyl and others. Most opioid drugs used to manage pain are addictive and subject to long-term abuse. Addiction is a chronic and relapsing brain disease characterized by a person pathologically pursuing reward and/or relief by substance use and other behaviors. In 2017, over 11 million people misused prescription opioids, and over 2 million people reported having OUD. Opioid overdose deaths in the U.S. exceeded 60,000 in 2017, and fentanyl-related overdose deaths increased by 45%. Drug overdose is the leading cause of accidental death in the US, with OUD driving this epidemic. Opioid overdose deaths drove down the US life expectancy over the last 3 years.

Medication-Assisted Treatment (MAT) is the use of FDA approved medications, in combination with counseling and behavioral therapies, to provide treatment of OUD. MAT is one of the major pillars of the federal response to the opioid epidemic in the US. Naltrexone, a commonly used drug to treat alcohol use disorders, is one of the 3 medications commonly used to treat opioid addiction. A consensus report issued in March, 2019 by the National Academy of Sciences concludes the MAT works and saves lives but is underused. Opioid antagonists, such as naltrexone, nalmefene, nalorphine, nalodeine, levallorphan, xorphanol, oxilorphan, samidorphan, 6-beta-naltrexol, methylnaltrexone, diprenorphine and cyprodime, act as competitive antagonists of opioid receptors, and they have the potential to block the euphoric and sedative effects of opioids. When used over time, they have the potential to reduce opioid cravings.

Despite the proven efficacy of MAT, medication adherence within OUD patients is poor; in one study, 50% of patients discontinued use of a long-acting naltrexone formulation after 6 weeks, and 85% discontinued use after 25 weeks. Other studies indicate a retention rate of <10% on naltrexone therapy after 4 months without incentives. Although extended release dosage forms of naltrexone (e.g., VIVITROL®, with a dosing period of 1 month) have produced higher rates of treatment compliance than daily oral dose forms, long-term patient retention rates remain low. Furthermore, longer-acting dose forms of naltrexone suffer from declining plasma levels of naltrexone and 6βnaltrexol (an active metabolite) over time. This may result in incomplete prophylaxis during the tail end of the nominal dosing period. For example, some patients receiving a series of VIVITROL™ injections for long-term treatment of OUD have been reported to overcome the opioid blockade of one injection prior to receiving another, resulting in acute symptoms of precipitated withdrawal.

Naltrexone and related opioid antagonists can be effective as part of MAT, but current oral and sustained release (depot) formulations have limited efficacy due to their short durations of action (<1 month) and poor PK profiles. The lack of longer-acting prophylactic pharmacologic options for OUD patients during maintenance therapy is an unmet medical need resulting in treatment relapses and low patient retention rates. Thus, there is a need for a delivery system that enables long term therapy for patients suffering with OUD. One solution could take the form of a subcutaneous, non-erodible implant that releases an opioid antagonist (e.g., naltrexone) from an internal reservoir by diffusion or controlled dissolution of a solid formulation. With respect to implantable systems, a delicate balance must be struck between interrelated structural elements of a delivery device. including its shape and size (which determine the dose of drug that can be loaded), the biocompatibility of the materials, and the diffusive surface area and its relative porosity. The output rate (i.e., mg of drug/day) must provide sufficient patient exposure after implantation as to provide the therapeutic effect (opioid antagonism) throughout a prolonged treatment period (e.g., 1-12 months). At the same time, there must be a sufficient mass of drug loaded within a device to support its target output rate and operative period, and yet the device cannot be too large as to become difficult to implant or remove, or create discomfort for the wearer. Questions of output rate and device size are complicated by the limited aqueous solubility of naltrexone and similar drugs in physiological fluids; depending on the device design, the pure drug may not dissolve at a sufficiently high rate to drive diffusion across a membrane of limited surface area—for instance, a circular membrane surface located at one or both termini of a cylindrical housing containing a formulation or aqueous suspension of a drug. Excipients that enhance the solubility of the drug within the device may increase the concentration gradient across device membranes and therefore increase the rate of drug diffusion from the implant, but at the expense of decreasing the drug load or increasing the overall device size. Biocompatibility of the materials becomes especially important due to both the length of the intended exposure period (e.g., 1-12 months), and the possibility of patients dropping out of treatment programs without having had their implants removed on schedule. Compositions, devices, and composition-device pairings that address these, and other, complications related to sustained and controlled delivery of small molecules to treat OUD are needed.

### BRIEF SUMMARY

The following aspects and embodiments thereof described and illustrated below are meant to be exemplary and illustrative, not limiting in scope.

In one aspect, a composition comprising an opioid antagonist and an organic monoprotic acid that (i) is present at a mole ratio of less than or equal to 1:1 with respect to the opioid antagonist, (ii) has a water solubility at room temperature of less than about 20 g/L, (iii) has a molecular weight of less than or equal to 400 g/mol, and (iv) maintains a pH of the opioid antagonist when hydrated to form an aqueous suspension that is between about 3.0-11.5.

In one aspect, a composition comprising an aqueous suspension is provided. The aqueous suspension comprises an opioid antagonist and an organic monoprotic acid that (i) is present at a mole ratio of less than or equal to 1:1 with respect to the opioid antagonist, (ii) has a water solubility at room temperature of less than about 20 g/L, (iii) has a molecular weight of less than or equal to 400 g/mol, and (iv) maintains a pH of the opioid antagonist when hydrated to form an aqueous suspension that is between about 3.0-11.5.

In one aspect, a composition comprising an aqueous suspension is provided. The aqueous suspension comprises an opioid antagonist and an organic dicarboxylic acid or tricarboxylic acid that (i) is present at a mole ratio of less than or equal to 0.5:1 or 0.333:1 with respect to the opioid antagonist, respectively, (ii) has a water solubility at room temperature of less than about 20 g/L, (iii) has a molecular weight of less than or equal to 600 g/mol, and (iv) maintains a pH of the opioid antagonist suspension in its environment of use that is between about 3.0-11.5.

In another aspect, a composition comprising an aqueous suspension is provided. The aqueous suspension comprises (a) an opioid antagonist as a major component expressed as a percentage of dry weight (>50%); (b) an organic acid that (i) is present at a mole ratio of less than or equal to 1:1 with respect to the opioid antagonist, (ii) has a water solubility at room temperature of less than about 20 g/L, (iii) has a molecular weight of less than or equal to 400 g/mol; and (c) a hydrophilic, non-hydrolyzable binder or a dispersant.

In another aspect, a composition comprising an aqueous suspension is provided. The aqueous suspension comprises (a) an opioid antagonist as the major component expressed as a percentage of dry weight (>50%); (b) an organic dicarboxylic acid that (i) is present at a mole ratio of less than or equal to 0.5:1 with respect to the opioid antagonist, (ii) has a water solubility at room temperature of less than about 20 g/L, (iii) has a molecular weight of less than or equal to 400 g/mol; and (c) a hydrophilic, non-hydrolyzable polymeric binder or a dispersant.

In one embodiment, the binder or dispersant is a polysaccharide, such as lactose or maltose, or a polymer, such as methylcellulose, hydroxypropylcellulose, hypromellose, polyvinylpyrrolidone (PVP), polyvinyl alcohol, crospovidone, or polyethyleneglycol (PEG).

In one embodiment, the opioid antagonist is a small molecule opioid antagonist. In one embodiment, the opioid antagonist is a competitive opioid antagonist. In one embodiment, the opioid antagonist is a base of an opioid antagonist. In another embodiment, the opioid antagonist is one or more of a small molecule opioid antagonist, a competitive opioid antagonist, and/or a base form of the opioid antagonist. In one embodiment, the base opioid antagonist is naltrexone or nalmefene.

In one embodiment, the aqueous suspension comprises, or is manufactured with, an organic acid suspended into a water-based solution, such as isotonic saline or an aqueous buffered solution.

In yet another embodiment, the aqueous suspension comprises the opioid antagonist suspended into a fractionated (i.e., cell-free), buffered physiological fluid. An example of such a fluid is filtered interstitial fluid. In a preferred embodiment, the aqueous suspension is prepared under conditions of dynamic exchange through a membrane.

In another embodiment, the aqueous suspension comprises in part, or is manufactured with, a pre-made salt formed between the small molecule therapeutic agent and the organic acid.

In another embodiment, the organic acid excipient and opioid antagonist are intimately mixed in a molar ratio of less than or equal to 1:1 by dissolution into a polar organic solvent such as methanol, ethanol, 1-propanol, 2-propanol, tert-butanol, acetone, 2-butanone, or ethyl acetate, followed by concentration of the intermediate solution to dryness.

In another embodiment, the acid and opioid antagonist are intimately mixed in a mole ratio of less than or equal to 1:1 (acid:opioid antagonist) in a polar organic solvent such as methanol, ethanol, 1-propanol, 2-propanol, tert-butanol, acetone, 2-butanone, or ethyl acetate such that concentration of the intermediate solution to dryness by evaporation in vacuo or by spray drying yields a solid consisting of an opioid antagonist and a salt form of the opioid antagonist. In a preferred embodiment, the resulting solid has an amorphous (non-crystalline) character.

In another embodiment, the acid and opioid antagonist are solid powders that are mixed in a mole ratio of less than or equal to 1:1 (acid:opioid antagonist) to assemble the formulation in a dry state. This dry formulation may also contain a dispersant or a binder as a minor component (as expressed by mass percentage).

In one embodiment, the solid powder composition comprising the opioid antagonist is fabricated into a shape, such as a tablet, pellet or a rod, sized for placement in the interior reservoir of the device. In one embodiment, the shape is a compressed shape.

In another embodiment, the composition of opioid antagonist is fabricated into a pellet or rod having an outer diameter that greater than or equal to about 95%, 96%, 97%, 98% or 99% or 99.5% of the inner diameter of the device's interior reservoir.

In one embodiment, the solid powder composition comprises a dispersant or a hydrophilic binder that facilitates fabrication of the composition into a compressed shape. In one embodiment, the compressed shape has a diameter greater than or equal to about 80%, 85%, 90%, 95%, 96%, 97%, 98% or 99% or 99.5% of the inner diameter of the device's interior reservoir. In other embodiments, the composition comprises between about 0.1-25% w/w, 0.5-20% w/w, 1-20% w/w, 2-20% w/w, 2-15% w/w, 2-12% w/w, 2-10% w/w, 0.5-10% w/w, 0.5-15% w/w, 0.5-8% w/w, 0.5-5% w/w, or 0.5-3% w/w hydrophilic binder or dispersant.

In one embodiment, the organic acid excipient is an aromatic carboxylic acid. Exemplary acids, in one embodiment, are those having a carboxylic acid group bound to an unsubstituted benzene or pyridine ring. In one embodiment, the carboxylic acid is selected from the group consisting of benzoic acid, picolinic acid, nicotinic acid, and isonicotinic acid.

In another embodiment, the carboxylic acid excipient is one having a benzene ring and between one and three electron-donating groups, such as amino, hydroxy, methyl, or methoxy. In another embodiment, the carboxylic acid has antioxidant properties.

In still another embodiment, the carboxylic acid is selected from the group consisting of o-anisic acid, m-anisic acid, p-anisic acid, p-aminobenzoic acid (PABA), o-aminobenzoic acid (anthranilic acid), o-toluic acid, m-toluic acid, p-toluic acid, salicylic acid, or vanillic acid.

In still another embodiment, the carboxylic acid is one having a carboxylic acid functional group separated from a benzene, pyridine, naphthalene, or quinoline ring by a chain of 1-4 saturated carbon atoms. In one embodiment, and by way of example, the carboxylic acid is phenylacetic acid or 3-phenylpropionic acid.

In another embodiment, the carboxylic acid is an aliphatic dicarboxylic acid with a 4-8 carbon chain separating the carboxylic acid groups. In one embodiment, and by way of example, the carboxylic acid is selected from the group consisting of adipic acid ((CH₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another embodiment, the carboxylic acid is an aromatic dicarboxylic acid such as phthalic, isophthalic, or terephthalic acid.

In another embodiment, the carboxylic acid is an unsaturated or polyunsaturated dicarboxylic acid containing 4-10 carbons. In one embodiment, and by way of example, the carboxylic acid is selected from the group consisting of fumaric acid, trans, trans-muconic acid, cis, trans-muconic acid, and cis,cis-muconic acid.

In other embodiments, the carboxylic acid is a cis-cinnamic acid or a trans-cinnamic acid. In still other embodiments, the carboxylic acid is a trans-cinnamic acid with one or two electron-donating groups selected from hydroxy, methoxy, amino, alkylamino, dialkylamino, or alkyl groups. In yet other embodiments, the trans-cinnamic acid is selected from the group consisting of o-coumaric acid, m-coumaric acid, p-coumaric acid, o-methylcinnamic acid, m-methylcinnamic acid, p-methylcinnamic acid, o-methoxycinnamic acid, m-methoxycinnamic acid, p-methoxycinnamic acid, and ferulic acid.

In another embodiment, the organic acid is a 1,3-dicarbonyl compound containing an acidic CH bond (pKa<8). In one embodiment, and by way of example, the organic acid is 2,2-dimethyl-1,3-dioxane-4,6-dione (Meldrum's acid), cyanuric acid, or barbituric acid.

In still another embodiment, the organic acid is an imide. In one embodiment, and by way of example, the imide is phthalimide or a substituted phthalimide. In another embodiment, the substituted phthalimide has at least one electron-withdrawing substituent.

In yet another embodiment, the organic acid is a hydroxamic acid. In one embodiment, and by way of example, the hydroxamic acid is an aromatic hydroxamic acid containing one hydroxamic functional group bonded directly to an aromatic ring. In one embodiment, the aromatic ring is selected from the group consisting of a benzene ring, a pyridine ring, a naphthalene ring, a quinoline ring, and a biphenyl ring. In still another embodiment, the hydroxamic acid is benzhydroxamic acid. In yet another embodiment, the hydroxamic acid is one containing a hydroxamic functional group separated from an aromatic ring by a chain of 1-4 sp³-hybridized carbon atoms.

In other embodiments, the hydroxamic acid is an aliphatic dihydroxamic acid containing 6-10 carbon atoms.

In another embodiment, the aromatic carboxylic acid is selected from the group consisting of 3-phenylpropionic acid, cinnamic acid, a hydroxy-derivative of cinnamic acid, a methoxy derivative of cinnamic acid, nicotinic acid, benzoic acid, an amino-derivative of benzoic acid, a methoxy derivative of benzoic acid, and phthalic acid.

In yet another embodiment, the hydroxy-derivative of cinnamic acid is m-coumaric acid or p-coumaric acid.

In yet other embodiments, the p-coumaric acid is trans-p-coumaric acid.

In other embodiments, the methoxy derivative of cinnamic acid is p-methoxycinnamic acid or m-methoxycinnamic acid.

In one embodiment, the composition is in a dry form. In another embodiment, the composition is compressed into one or more pellets prior to being mixed with a sterile or filtered aqueous fluid.

In another aspect, a device comprising a composition as described herein is provided. The device is configured for subcutaneous implantation into a mammal.

In another aspect, an implantable device is provided. The device comprises a reservoir containing a formulation of an opioid antagonist and an organic monoprotic acid in a mole ratio of less than or equal to 1:1 (acid:opioid antagonist) sufficient to provide substantially zero-order release of the opioid antagonist for a delivery period of at least about 30 days and at a rate that provides a therapeutic effect.

In another aspect, an implantable device is provided. The device comprises a reservoir containing a formulation of an opioid antagonist, the formulation comprising (i) an amount of the opioid antagonist sufficient to provide substantially zero-order release of the opioid antagonist for a delivery period of at least about 30 days and at a rate that provides a therapeutic effect and (ii) an organic monoprotic acid at a molar ratio of less than or equal to 1:1 relative to the opioid antagonist.

In another aspect, an implantable device is provided. The device comprises a reservoir containing a formulation of an opioid antagonist, the formulation comprising (i) an amount of the opioid antagonist sufficient to provide substantially zero-order release of the opioid antagonist for a delivery period of at least about 30 days and at a rate that provides a therapeutic effect and (ii) an organic diprotic or triprotic acid at a molar ratio of less than or equal to 0.5:1 or 0.333:1 relative to the opioid antagonist, respectively.

In one embodiment, the formulation is in dry form. In various embodiments, and by way of example, the formulation is a powder, a tablet or a film; or a mixture of two or more powders, tablets, or films.

In another embodiment, the formulation hydrates in the presence of a sterile or filtered aqueous solution to form an aqueous suspension.

In another embodiment, the opioid antagonist is released from the device at a rate that provides a therapeutic effect for the treatment period.

In still another embodiment, the organic monoprotic acid excipient is present in a mole ratio of less than or equal to 1:1 relative to the opioid antagonist and has a water solubility at room temperature of less than about 20 g/L. In still another embodiment, the organic acid has a water solubility at room temperature between 0.1 and 10 g/L and a molar mass less than 400 grams per mole.

In another embodiment, the organic acid excipient present in a mole ratio of less than or equal to 1:1 (relative to the opioid antagonist) has a water solubility at room temperature of less than about 20 g/L and a pKa between 3 and 6. In another embodiment, the organic acid has a water solubility at room temperature between 0.1 and 10 g/L, a molar mass less than 400 grams per mole, and a pKa between 3 and 6.

In another embodiment, two or more organic acid excipients, each with a water solubility of 0.1 to 10 g/L, a molar mass less than 400 grams per mole, and a pKa between 3 and 6 are used in combination, such that the total number of acid equivalents (i.e., carboxylic acid functional groups) is less than or equal to 1:1 (relative to the drug).

In another aspect, a method for sustained, controlled delivery of an opioid antagonist is provided, where the method comprises providing a composition or a device as described herein. In some embodiments, the method further comprises administering the device, such as by subcutaneous implantation.

In another aspect, a method to provide maintenance therapy to treat opioid use disorder or alcohol abuse disorder is provided, where the method comprises providing a composition or a device as described herein. In some embodiments, the method further comprises administering the device, such as by subcutaneous implantation.

In addition to the exemplary aspects and embodiments described above, further aspects and embodiments will become apparent by reference to the drawings and by study of the following descriptions.

Additional embodiments of the present methods, devices and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are illustrations of a drug delivery device, in assembled form (FIG. 1A) and in unassembled form (FIG. 1B).

FIGS. 1C-1F illustrate a portion of a first exemplary drug delivery device, showing the end cap subassembly in cross section when fully assembled (FIG. 1C) and in an exploded view (FIG. 1D), and in isometric view when assembled (FIG. 1E). FIG. 1F shows an exploded view of the cap subassembly alone. The numbered elements of the subassembly are 1=cap, 2=porous membrane, 3=seal, 4=retention ring, and 5=drug device reservoir.

FIGS. 1G-1K illustrate a portion of a second exemplary drug delivery device, showing the end cap subassembly in cross section when fully assembled (FIG. 1G) and in an exploded view (FIG. 1H), and in isometric view when assembled (FIG. 14 FIGS. 1J-1K show an assembled and exploded view of the cap subassembly alone. The numbered elements of the subassembly are 1=cap, 2=porous membrane, 3=seal, 4=drug delivery device reservoir, and 5=retention ring.

FIG. 2 is a graph showing the cumulative in vitro release of naltrexone in mg, as a function of time, in days, from drug delivery devices with a diffusive surface area ranging from 5 to 20 mm² and comprising aqueous formulations of anisic acid and naltrexone base in molar ratios of 0:1 (squares); 1:1 (triangles, open squares) in the form of compressed tablets; and 1:1 (open diamonds) in the form of a powder. A device loaded with naltrexone HCl (triangles) also tested for comparison, and set of devices device containing a 1:1 naltrexone base/anisic acid formulation had a reduced membrane surface area of about 25% (circles).

FIG. 3 is a graph showing the cumulative in vitro release of naltrexone in mg, as a function of time, in days, from drug delivery devices comprising compressed pellets naltrexone base (diamonds) or naltrexone base and p-anisic acid at a mole ratio of 50% or 100% relative to naltrexone (diamonds, circles) or p-aminobenzoic acid at a mole ratio of 25% or 50% relative to naltrexone (x symbols and * symbols).

FIG. 4 is a graph showing the weight-normalized plasma levels of naltrexone, in ng/mL, in rats (n=5) implanted with drug delivery devices comprising a compressed formulation of naltrexone base and p-anisic acid in a 1:1 mole ratio, as a function of time in days after implantation, over a period of approximately 100 days.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein in reference to methods of administration of a small molecule which reduces the frequency of, or delays the onset of, symptoms of a medical condition (e.g., opioid use disorder or alcohol abuse disorder) in a subject relative to a subject not receiving the compound or composition. This can include reversing, reducing, or arresting the symptoms, clinical signs, and underlying pathology of a condition in a manner to improve or stabilize a subject's condition (e.g., controlling opioid or alcohol addiction).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Formulations to Enhance the Solubility of an Opioid Antagonist

In one aspect, a composition or formulation is provided, in which a stoichiometric excess of an opioid antagonist is wholly or partially solubilized through the use of one or more partially soluble organic acids to improve delivery of the therapeutic agent from a device or drug delivery platform for a sustained period of time. In one embodiment, the composition is an aqueous suspension or slurry. In another embodiment, the composition is a heterogeneous or non-uniform mixture containing more than one phase. The solution, suspension, or mixture can be, in some embodiments, an aqueous mixture or an aqueous heterogeneous mixture. For example, the composition may comprise, consist essentially of, or consist of an aqueous suspension of the opioid antagonist and an organic acid, or the opioid antagonist and a salt form of the opioid antagonist, or the opioid antagonist, an organic acid, and a salt form of the opioid antagonist, each in a partially dissolved state. In another embodiment, the composition is in dry form (e.g., lyophilized, spray dried, desiccated, etc.).

In these various embodiments, the composition comprises an opioid antagonist that can function as a Bronsted or Lewis base and an organic acid that has one or more of the following characteristics: (i) it has a water solubility at room temperature (e.g., approximately 25° C.) of less than about 20 or more preferably, of between about 0.1 to 10 g/L; (ii) it has a molar mass less than or equal to 400 grams per mole; (iii) it is present at a molar ratio of less than or equal to 1:1 relative to the therapeutic agent; and (iv) it maintains a pH of the suspension (or solution) in its environment of use that is between about 3.0-11.5, for a period of at least about 30 days. The composition may additionally comprise a sterile or filtered aqueous fluid, for example water, isotonic saline, buffer or a water-solvent mixture.

In embodiments where the composition is in dry form, the aqueous fluid hydrates the composition, for example, in situ in its environment of use or prior to use. The dry form of the composition may be compressed to form a solid, compressed shape. In one embodiment, the compressed shape is a tablet, a pellet or a rod. As can be appreciated, a tablet is a disc-shaped object; a pellet is a spherical or ovoid shaped object, and a rod has an cylindrical, somewhat elongate shape. In an embodiment, the compressed shape has a density exceeding 1.00 g/cm³. In one embodiment, the pellets or rods are non-porous. In another embodiment, the composition is molded or compressed to form a tablet, pellet or rod with a density of between about 1.00 g/cm³-1.20 g/m³.

As noted above, the formulations described herein provide a wholly or partially solubilized opioid antagonist in order to permit its delivery for a sustained period. In one embodiment, a sustained period of time intends a period of at least about two weeks to about twelve months, or at least about four weeks to six months.

Also as noted above, for the formulations described herein the opioid antagonist is made sufficiently soluble in part by maintaining a particular pH range of the formulation in its environment of use for the stated period of time. In one embodiment, the environment of use is in vivo. For example, the formulation may be part of a drug delivery device that is implanted in vivo and several examples of such devices are provided below. In another embodiment, the environment of use is in vitro in a release medium maintained at about 37° C.

The components of the composition, namely the opioid antagonist and the organic acid, are now described.

### A. Opioid Antagonist

In one embodiment, the compositions comprise an opioid antagonist. The opioid antagonist is, in some embodiments, a small molecule compound, meaning a compound with a molecular weight of less than about 450 g/mol. In one embodiment, the small molecule opioid antagonist has a molecular weight of between about 250-450 g/mol.

In other embodiments, the compositions comprise an opioid antagonist selected from naloxone (327.3 g/mol), nalmefene (339.4 g/mol), nalorphine (311.4 g/mol), nalodeine (325.4 g/mol), levallorphan (283.4 g/mol), xorphanol (337.5 g/mol), oxilorphan (313.4 g/mol), samidorphan (370.4 g/mol), 6-beta-naltrexol (343.4 g/mol), methylnaltrexone (356.4 g/mol), diprenorphine (425.6 g/mol), and/or cyprodime (355.5 g/mol). In other embodiments, the compositions comprise a mixture of two or more opioid antagonists.

In one embodiment, the opioid antagonist (i) has a limited solubility in physiological fluids, and (ii) functions as a Bronsted or Lewis base. As will be described below, in the presence of an aqueous fluid and an organic acid excipient that (i) is present at a mole ratio of less than or equal to 1:1 with respect to the drug, (ii) has a water solubility at room temperature of less than about 20 g/L, and (iii) has a molecular weight of less than or equal to 400 g/mol, a suspension or slurry is produced with a pH (within the aqueous fraction) of about between 3.0-11.5. Within the aqueous phase of this suspension or slurry, the concentration of soluble drug (expressed as the sum of its protonated and deprotonated forms) is greater than the concentration of the drug in the absence of the excipients.

The terms drug, therapeutic agent and opioid antagonist are used interchangeably herein.

### B. Organic Acids

The composition, in addition to the opioid antagonist therapeutic agent, comprises an organic acid or combination of organic acids. The organic acid is one that has one or more of the following features: (i) a water solubility at room temperature of between 0.1 and 10 g/L, between 0.1 and 20 g/L, or of less than about 20 g/L; (ii) a molar mass less than 400 grams per mole or of between 1-400 grams per mole; (iii) is present at a mole ratio of less than or equal to 1:1 with respect to the therapeutic agent (or less than or equal to 0.5:1 or 0.333:1 for a diprotic or triprotic acid, respectively); and/or (iv) maintains a pH of the aqueous suspension or solution in its environment of use approximately equal to or less than 11.5, 11, 10.5, 10, 9.5, 9, or 8.5 for a period of at least about 30 days. In some embodiments, the organic acid is a monoprotic acid which is present in the composition and/or in the aqueous suspension or solution at an organic acid:therapeutic agent mole ratio of equal to or less than 1:1, 0.95:1, 0.9:1, 0.85:1, 0.8:1, 0.75:1, 0.7:1, 0.65:1, 0.6:1, 0.551, 0.5:1, 0.45:1, 0.4:1, 0.35:1, 0.3:1, 0.25:1, 0.2:1. 0.15:1, or 0.1:1, inclusive of any ranges constructed from any of these discrete ratios, such as a range of 0.1:1 to 1:1, 0.25:1 to 0.9:1, 0.25:1 to 0.8:1, etc. In another embodiment, the organic acid is a diprotic acid that is present in the composition and/or in the aqueous suspension or solution at an acid:therapeutic agent mole ratio equal to or less than 0.5:1, 0.45:1, 0.4:1, 0.35:1, 0.3:1, 0.25:1, 0.2:1. 0.15:1, or 0.1:1, inclusive of any ranges constructed from any of these discrete ratios, such as a range of 0.1:1 to 0.5:1, 0.15:1 to 0.45:1, 0.2:1 to 0.5:1, etc. In another embodiment, the organic acid is a triprotic acid that is present in the composition and/or in the aqueous suspension or solution at an acid:therapeutic agent mole ratio equal to or less than 0.33:1, 0.32:1, 0.31:1, 0.3:1, 0.29:1, 0.28:1, 0.27:1, 0.26:1, 0.25:1, 0.2:1. 0.15:1, or 0.1:1, inclusive of any ranges constructed from any of these discrete ratios, such as a range of 0.1:1 to 0.33:1, 0.15:1 to 0.3:1, 0.1:1 to 0.25:1, etc. It will be appreciated that the formulation may comprise a combination of a monoprotic organic acid, a diprotic organic acid, and/or a triprotic organic acid.

The environment of use, in one embodiment, is an in vivo environment. In an embodiment, the in vivo environment is subcutaneous site. In an embodiment, the in vivo environment is subcutaneous site in a human subject, wherein the site is maintained at essentially body temperature. The environment of use, in another embodiment, is an in vitro environment.

As described above, the compositions enhance the solubility of the small molecule therapeutic agent, permitting use of the composition in a drug delivery platform that provides sustained release for an extended period of time. Examples of organic acids for use in the compositions are now described.

In a first embodiment, the organic acid is a carboxylic acid. Examples include aromatic carboxylic acids where a carboxylic acid group is bonded directly to an aromatic ring. For example, the aromatic carboxylic acid can have one carboxylic acid group bound to an unsubstituted benzene or pyridine ring. Examples include benzoic acid, picolinic acid, nicotinic acid, or isonicotinic acid. In another example, the aromatic carboxylic acid is one having a benzene ring and one electron-donating group with antioxidant properties. Specific examples include o-anisic acid, m-anisic acid, p-anisic acid, p-aminobenzoic acid (PABA), o-aminobenzoic acid (anthranilic acid), o-toluic acid, m-toluic acid, p-toluic acid, salicylic acid.

In yet another example, the aromatic carboxylic acid is one having a single benzene ring and two electron donating groups with antioxidant properties. A specific example is vanillic acid. In still another example, the aromatic carboxylic acid is one having two or more carboxylic acid groups bonded to a benzene ring. Specific examples include phthalic acid and isophthalic acid.

In another example, the aromatic carboxylic acid is one having one carboxylic acid group bonded to a naphthalene or quinoline ring. Examples include 1-naphthoic acid, 2-naphthoic acid, quinaldic acid, 3-quinolinecarboxylic acid, 4-quinolinecarboxylic acid, 5-quinolinecarboxylic acid, 6-quinolinecarboxylic acid, 7-quinolinecarboxylic acid, and 8-quinolinecarboxylic acid. A further grouping of acids of this type, with one carboxylic acid group bonded to a naphthalene or quinoline ring, include those containing at least one additional electron-donating group, such as a hydroxy, methoxy, amino, alkylamino, dialkylamino, or alkyl group. Examples of acids in this grouping include 6-hydroxy-2-naphthoic acid, 6-hydroxy-3-naphthoic acid, 8-hydroxy-2-quinolinecarboxylic acid, 8-hydroxy-7-quinolinecarboxylic acid, and isomers of each.

In another exemplary embodiment, the carboxylic acid is one having one or two carboxylic acid groups bonded to a biphenyl ring system. Examples of such acids include biphenyl-4-carboxylic acid and diphenic acid. Other acids in this group include those that incorporate one or more electron donating substituents on the ring system in addition to the carboxylic acid group or groups. Examples include 4′-hydroxy-4-biphenylcarboxylic acid, 4′-hydroxy-2-biphenylcarboxylic acid, 4′-methyl-4-biphenylcarboxylic acid, 4′-methyl-2-biphenylcarboxylic acid, 4′-methoxy-4-biphenylcarboxylic acid, and 4′-methoxy-2-biphenylcarboxylic acid.

In another exemplary embodiment, the acid is a di- or tri-carboxylic acid having two or three carboxylic acid groups bonded to a naphthalene or quinoline ring. Examples include 1,4-naphthalenedicarboxylic acid and 2,6-naphthalenedicarboxylic acid.

In another exemplary embodiment, the carboxylic acid is one having a carboxylic acid functional group separated from a benzene, pyridine, naphthalene, or quinoline ring by a chain of 1-4 saturated carbon atoms. Examples of acids in this embodiment include phenylacetic acid and 3-phenylpropionic acid.

In another exemplary embodiment, the carboxylic acid is an aliphatic dicarboxylic acid with 6-10 carbon atoms, such as adipic acid ((CH₂)₄(COOH)₂), pimelic acid (HO₂C(CH₂)₅CO₂H), suberic acid (HO₂C(CH₂)₆CO₂H), azelaic acid (HO₂C(CH₂)₇CO₂H), and sebacic acid (HO₂C(CH₂)₈CO₂H).

In another exemplary embodiment, the carboxylic acid is an unsaturated or polyunsaturated dicarboxylic acid containing 4-10 carbons. Examples of acids in this embodiment include fumaric acid, trans, trans-muconic acid, cis, trans-muconic acid, and cis, cis-muconic acid.

In another exemplary embodiment, the carboxylic acid is a cis- or trans-cinnamic acid. In one embodiment, the trans-cinnamic acid has one or two electron-donating groups selected from hydroxy, methoxy, amino, alkylamino, dialkylamino, or alkyl groups. Examples include o-coumaric acid, m-coumaric acid, p-coumaric acid, o-methylcinnamic acid, m-methylcinnamic acid, p-methylcinnamic acid, o-methoxycinnamic acid, m-methoxycinnamic acid, and p-methoxycinnamic acid, and ferulic acid.

In another embodiment, the organic acid is a phenol or a naphthol substituted with between about 2-5 electron-withdrawing groups selected from —F, —Cl, —Br, —I, —CN, —CHO (aldehyde), —COR (ketone), and NO₂. Examples include 2,4-dinitrophenol.

In another embodiment, the organic acid is a 1,3-dicarbonyl compound containing an acidic CH bond (pKa<9). Examples include 2,2-dimethyl-1,3-dioxane-4,6-dione (Meldrum's acid), cyanuric acid, or barbituric acid.

In another embodiment, the organic acid is an imide, such as phthalimide. In one embodiment, the phthalimide is substituted with at least one electron-withdrawing substituent.

In another embodiment, the organic acid is a hydroxamic acid. The hydroxamic acid may be, in some embodiments, an aromatic hydroxamic acid containing one hydroxamic functional group bonded directly to an aromatic ring. The aromatic ring is selected from the group consisting of a benzene ring, a pyridine ring, a naphthalene ring, a quinoline ring, and a biphenyl ring. Examples include benzhydroxamic acid. The hydroxamic acid can also be one containing a hydroxamic functional group separated from an aromatic ring by a chain of 1-4 sp^{a}-hybridized carbon atoms. Dihydroxamic acids containing two or more hydroxamic acid functional groups bonded directly to a benzene, pyridine, naphthalene, quinoline, or biphenyl ring system are also contemplated. In addition, substituted derivatives of the hydroxamic acids described above that contain electron donating substituents such as hydroxy, methoxy, amino, alkylamino, dialkylamino, or alkyl groups are contemplated. Also contemplated are aliphatic dihydroxamic acids containing 6-10 carbon atoms, such as suberohydroxamic acid, and unsaturated dihydroxamic acids containing 6-10 carbon atoms.

The organic acids for use in the compositions described herein are preferably those with a water solubility at room temperature between 0.1 and 10 g/L or, alternatively, of less than about 20 g/L. In another embodiment, the organic acids for use in the compositions described herein have a molar mass less than 400 grams per mole. In another embodiment, the organic acids for use in the compositions described herein are non-polymeric or non-oligomeric. In another embodiment, the organic acids for use in the compositions described herein do not have a polymeric or oligomeric backbone and/or are not attached to a polymeric or oligomeric backbone. In another embodiment, the acid has a water solubility at room temperature of less than about 20 g/L and a pKa value between about 3 and 6, more preferably a pKa value of between about 3-5.5 or between about 3.5-5.5. In other embodiments, the organic acid is crystalline and has a melting temperature of more than about 37° C.

Compositions comprising an opioid antagonist and an organic acid present at a mole ratio of less than or equal to 1:1 with respect to the opioid antagonist are prepared by mixing the organic acid and the opioid antagonist together in a suitable solvent. In some embodiments, the solvent is a polar solvent that facilitates conversion of some fraction of the opioid antagonist into a salt form by reaction with the organic acid. In some embodiments, the solvent is an aqueous fluid, such as a buffer or a water-organic solvent mixture.

In embodiments where the composition is within a reservoir of a drug delivery device, it will be appreciated that the device when placed in its environment of use is open to the environment of use. That is, the environment of use and the composition in the device are in fluid communication via the pore or porous membrane in the drug delivery device. The compositions described herein include suspensions or slurries that contain the free base form of an opioid antagonist (e.g., naltrexone) in combination with a salt form of the drug. Both components are initially present at concentrations greatly exceeding their saturation points. It will also be appreciated that the opioid antagonist and its salt constitute a conjugate acid-base pair, and therefore, the fluid compartment inside of the device will be buffered. Therefore, in accordance with aforementioned embodiments, the slow dissolution of opioid antagonist base and opioid antagonist salt within the reservoir maintains a relatively constant pH within the aqueous phase of the device interior over the operative period of the device. As the solubility of opioid antagonists decreases with increasing pH, preferable compositions maintain the desired pH of the suspension or heterogeneous solution at a value between about 3.0-11.5. In one embodiment, the opioid antagonist is naltrexone. In one embodiment, the pH of the suspension or heterogeneous solution is between about 4.5 and 10.6. In another embodiment, the pH of the suspension or heterogeneous solution is between about 3.5-11.0, 4.0-11.0, 4.75-10.75 or 4.6-10.7.

It will also be appreciated that physiological fluids are buffered at near-neutral pH (7.4), and that the presence of mild physiological acids may be sufficient to dissolve many weakly basic opioid antagonists to an appreciable extent. In yet another embodiment, the aqueous suspension comprises the opioid antagonist suspended into a fluid in communication with a physiological buffer (e.g., interstitial fluid) through a porous membrane. In this case, the membrane surface area and porosity are selected to maintain a pH within the aqueous phase of the device interior at a value between 7.4 and 10.6 through the constant addition of endogenous buffering acids (e.g., carbonic acid and bicarbonate) and subtraction of dissolving opioid antagonist from the device by diffusion.

### C. Delivery Devices

In one embodiment, the drug delivery device is one having a housing member that defines a reservoir in which the compositions and/or the aqueous suspensions described above is retained. The housing member is of a size and shape that is suitable for implantation into the body. A cylindrical shape is preferable for subcutaneous implantation using a cannula or trocar. The outer diameter of a cylindrically shaped housing member would preferably be in the range of 2 mm to 6 mm and the length in the range of about 10 mm to about 50 mm. The composition or aqueous suspension, in one embodiment, is initially present in a dry form within the reservoir of the device. For example, an aqueous suspension comprising an organic acid and the opioid antagonist at a molar ratio of less than or equal to 1:1 is prepared and subsequently spray dried, milled or lyophilized to provide a dried form of the aqueous suspension. Alternatively, the individual components in dried form—i.e., the therapeutic agent as a dry solid and the organic acid as a dry solid—are physically mixed (blended) in the correct proportions to provide upon later hydration the desired aqueous suspension. Alternatively, the therapeutic agent and the organic acid may be co-dissolved within a suitable organic solvent such as methanol, ethanol, 1-propanol, 2-propanol, tert-butanol, acetone, 2-butanone, or ethyl acetate, followed by concentration in vacuo or spray drying to yield a homogeneous powder suitable for resuspension into an aqueous medium. Any of the aforementioned solid compositions can be tableted or pelleted, loaded in the device and hydrated in situ upon subcutaneous implantation of a device containing the dried composition, or more preferably, the composition can be hydrated at the time of subcutaneous implantation by a clinician introducing a liquid (e.g. a physiological buffer, isotonic saline, phosphate buffered saline, or aqueous propylene glycol) to a reservoir or matrix containing the composition. The liquid can be provided as part of a kit comprising the drug delivery device and a vial comprising a hydration liquid.

The interior volume of the implantable device is sufficient to hold a quantity of opioid antagonist formulation sufficient to dose a human subject for the intended therapeutic period. The therapeutic period may range from about 1 to 12 months, and preferably, is greater than about 2 months, 3 months, 4 months or 6 months and, optionally, less than about 14 months or about 12 months. In a preferred embodiment, the device has an interior volume of between about 500 μL and 1000 μL.

The interior diameter of the implantable drug delivery device is constant throughout its length, and, in one embodiment, the diameter of the interior reservoir is not more than 5% greater than the diameter of the solid, compressed shape (e.g., tablets, pellets or rods) that is/are placed into the interior reservoir. In other embodiments, the diameter of the interior reservoir 5%, 4.4%, 4%, 3.5%, 3%, 2.5%, 2%, 1.5%, 1% or 0.5% greater than the diameter of the solid, compressed shape(s) to be placed into the interior reservoir. This configuration permits only a small amount of fluid to penetrate between the wall of the interior reservoir and the curved, outer surface of the compressed shape. A limited fluid volume limits the number of diffusion paths (and hence, the absolute diffusion rate) of particles exiting from the curved surface of the compressed shape relative to particles exiting from the orthogonal flat end of the compressed shape that is aligned with the porous membrane.

The compressed shape within the device may be hydrated in situ upon subcutaneous implantation of the device, or more preferably, the compressed shape can be hydrated immediately prior to subcutaneous implantation by a clinician introducing a liquid (e.g., a physiological buffer, isotonic saline, phosphate buffered saline, or aqueous propylene glycol) to the reservoir. The reservoir may be prepared and sealed under vacuum within a secondary container (for instance, a lyophilization vial sealed with a soft rubber septum) to facilitate this process. The liquid can be provided as part of a kit comprising a vial bearing the drug delivery device and a second vial bearing the hydration liquid.

In another embodiment, the device reservoir material is titanium, the length of the reservoir is between about 40-50 mm, the outer diameter of the reservoir is about 4-5 mm, the porous partition is an about 0.1 micron pore size polyvinylidene difluoride (PVDF) membrane with a diffusive surface area of between about 2-22 mm², and the formulation is a mixture of anisic acid and naltrexone base in a molar ratio of less than or equal to 1:1.

In yet another embodiment, the device reservoir material is titanium, the length of the reservoir is between about 45-50 mm, the outer diameter of the reservoir is between about 4-5 mm, the porous partition is an about 0.1 micron pore size polyvinylidene difluoride (PVDF) membrane with a diffusive surface area of between about 20-25 mm², and the formulation is naltrexone base, alone or with a hydrophilic dispersant as a minor component as measured by % mass.

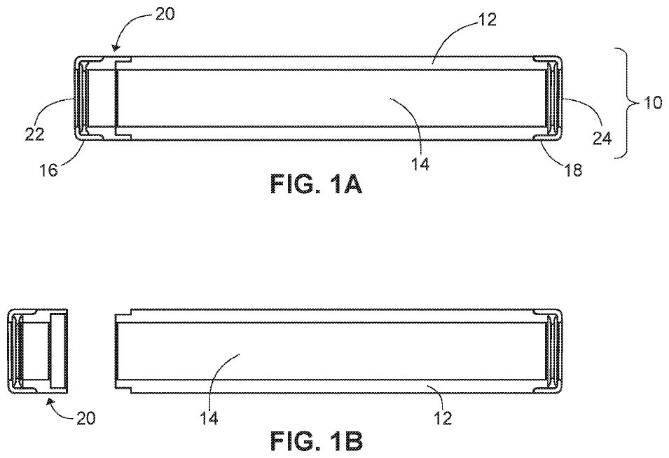

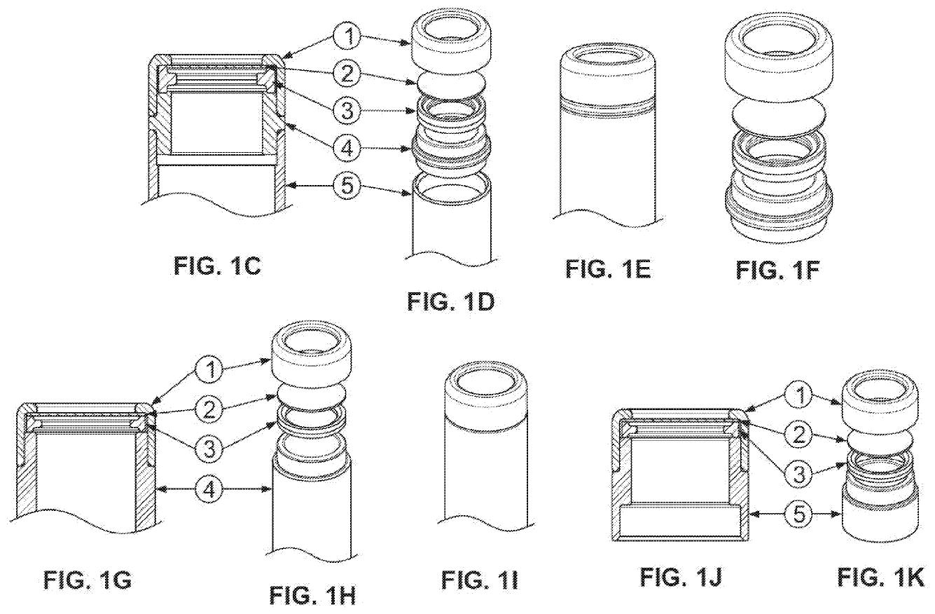

An example of a drug delivery device is provided in FIGS. 1A-1B, FIG. 1A illustrates a device 10, assembled and ready for implantation, in an anatomical compartment of a subject, such as under the skin or in the peritoneal cavity. The device is comprised of a non-erodible housing member 12 that defines an internal compartment or reservoir 14. Contained within the reservoir is a composition or formulation as described herein. Housing member 12 has first and second ends, 16, 18. First end 16 is sealed with a fluid-tight end-cap 20, seen best in FIG. 1B that illustrates device 10 in its unassembled form. End cap 20 may optionally comprise a porous membrane or semi-permeable membrane or porous partition 22. Second end 18 is optionally fitted with a porous membrane, semi-permeable membrane, or porous partition 24. It will be appreciated that a porous partition, porous membrane, or semi-permeable membrane can be situated or disposed anywhere on the housing member for fluid communication with the reservoir, for example, the partition or membrane can be seated or secured on the wall that extends between the first and second ends. The example herein with the partition or membrane in one or both ends is merely exemplary.

FIGS. 1C-1K illustrate the end caps and end cap subassembly portions of the exemplary drug delivery devices. The numbered elements of the subassembly illustrated in FIGS. 1C-1F are 1=cap, 2=porous membrane, 3=seal, 4=retention ring, and 5=drug device reservoir. The numbered elements of the subassembly illustrated in FIGS. 1G-1K are 1=cap, 2=porous membrane, 3=seal, 4=drug delivery device reservoir, and 5=retention ring.

The device interior contains a formulation comprising a opioid antagonist drug that is i) poorly soluble in aqueous physiological fluids and/or ii) can function as a Bronsted or Lewis base. The formulation also comprises an organic acid at a molar ratio of less than or equal to 1:1 with respect to the opioid antagonist, where the acid (i) has a solubility in water between 0.1 and 10 g/L, or of less than or equal to 20 g/L at 25° C., and (ii) dissolves at least partially in the presence of the opioid antagonist and a physiological buffer, to produce a suspension or slurry of protonated and deprotonated opioid antagonist with a pH (within the aqueous fraction) that is between about 3.0-11.5.

As used herein, the terms “porous membrane” and “porous partition” intend a structural member that has a plurality of pores in the nanometer or micrometer (μm) range, preferably in range of about 0.1-0.45 μm. The drug (opioid antagonist) elutes from the device as is diffuses across the partition; diffusion, and thus the rate of drug release, is directly proportional to the surface area of the porous partition. In order to deliver a therapeutic dose from the device the surface area of the partition is about 2.5-22 mm² total per device. The porous partition permits passage of the therapeutic agent (opioid antagonist) in a soluble form from the formulation contained within the reservoir. The porous partition can also permit passage of the organic acid that is part of the formulation in its soluble form. The porous partition in a preferred embodiment retains the therapeutic agent and/or the organic acid in their insoluble forms. That is, the therapeutic agent and/or the organic acid in insoluble form preferably do not pass through the pores of the porous partition. The drug delivery device is described in detail in U.S. 2011/0106006, which is incorporated by reference herein. In one embodiment, the membrane/partition has a porosity of greater than or equal to 70%. In another embodiment, the membrane/partition has a porosity of greater than or equal to 50%.

Studies were conducted to evaluate the release rate and kinetic order of release from drug delivery devices containing in the device reservoir compositions comprised of and opioid antagonist and organic acid, using as models naltrexone and anisic acid. The study is described in Example 1.

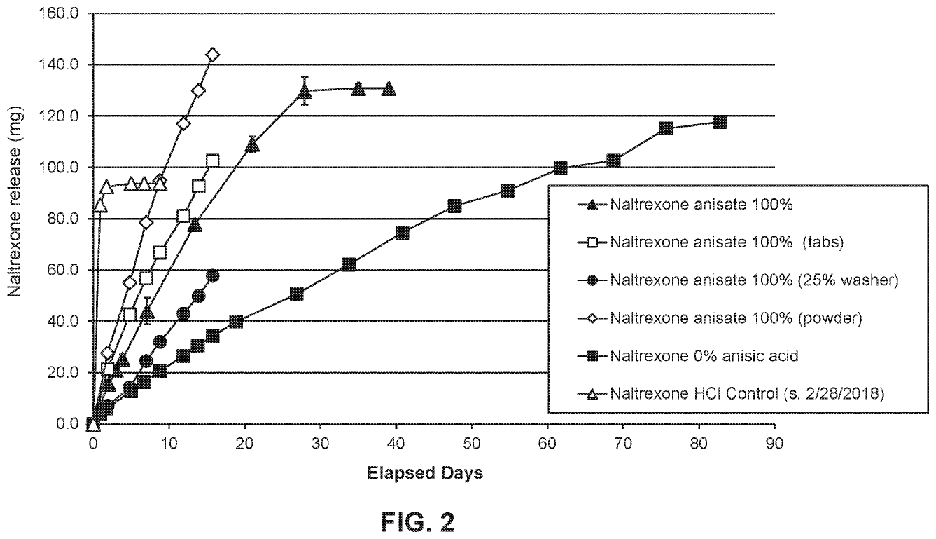

FIG. 2 shows cumulative release of naltrexone, in mg, as a function of time, in days, from drug delivery devices comprising aqueous formulations of anisic acid and naltrexone in molar ratios of 0:1 (squares); 1:1 tablets (triangles, open squares); and 1:1 powder (open diamonds). A device loaded with naltrexone HCl (triangles) was included in the study as a control. In one set of devices containing a 1:1 naltrexone/anisic acid formulation, the membrane surface area was reduced by about 25% (circles). The addition of an organic acid, anisic acid, to the formulation increased the release rate of therapeutic agent with near zero-order kinetics for the delivery period. A reduction in membrane surface area by approximately 75% produced a reduction in output rate (˜40%) for systems loaded with the 1:1 naltrexone/anisic acid formulation. Note that the curves plateau as the devices runs out of drug.

The data from this study shows that drug delivery devices comprising a porous membrane with between about 5-20 mm² diffusible area and approximately 70% porosity release naltrexone with no additional anisic acid eluted at 1.5 mg/day in vitro, whereas the naltrexone HCl control eluted over 50 mg/day. Naltrexone-anisic acid 1:1 (tablets) eluted at a rate of between about 4.6 to 6.3 mg/day and the 1:1 powder formulation eluted at a rate of about 9 mg/day. A configuration with a total diffusible surface area of about 5 mm² and a naltrexone-anisic acid mole ratio of 1:1 eluted at about 3.6 mg/day.

In another study, detailed in Example 2, compressed pellets were prepared from mixtures of naltrexone base, an organic acid excipient, polyvinylpyrrolidone and stearic acid. The organic acid excipients were p-anisic acid or p-aminobenzoic acid, and were included in the mixtures at a mole ratio or 25%, 50%, or 100% relative to naltrexone based. Compressed tables of naltrexone base, polyvinylpyrrolidone and stearic acid were prepared as a control. Drug delivery devices were filled with the compressed pellets and the in vitro release of naltrexone was measured.

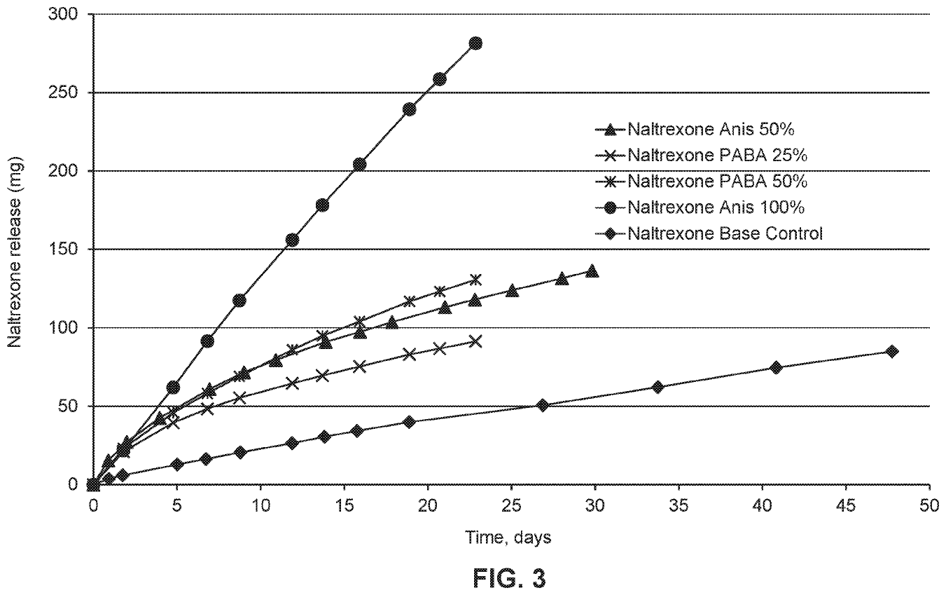

FIG. 3 shows the cumulative in vitro release of naltrexone in mg, as a function of time, in days, from drug delivery devices comprising compressed pellets naltrexone base (diamonds) or naltrexone base and p-anisic acid at a mole ratio of 50% or 100% relative to naltrexone (diamonds, circles) or p-aminobenzoic acid at a mole ratio of 25% or 50% relative to naltrexone (x symbols and * symbols). Devices comprising a formulation that included an organic acidic excipient that partly converts naltrexone base into a salt form of the drug elute drug at an enhanced rate relative to the devices with a formulation lacking the organic acid excipient (i.e., with the drug in its base form).

An in vivo study was conducted, as described in Example 3. Drug delivery devices comprising solid, dry pellets of naltrexone base and p-anisic acid in a mole ratio of 1:1 were prepared. The devices were implanted into rats (n=5). Plasma samples and animal weights were obtained at time points over a period of approximately 100 days, and plasma concentrations of naltrexone were determined, as described in Example 3. At the end of the study, the devices were recovered and subjected to mass balance analysis. Residual naltrexone was quantified and compared to the initial quantity loaded for each device. Based on this analysis, test devices eluted naltrexone at an average rate of approximately 2.5 mg/day.

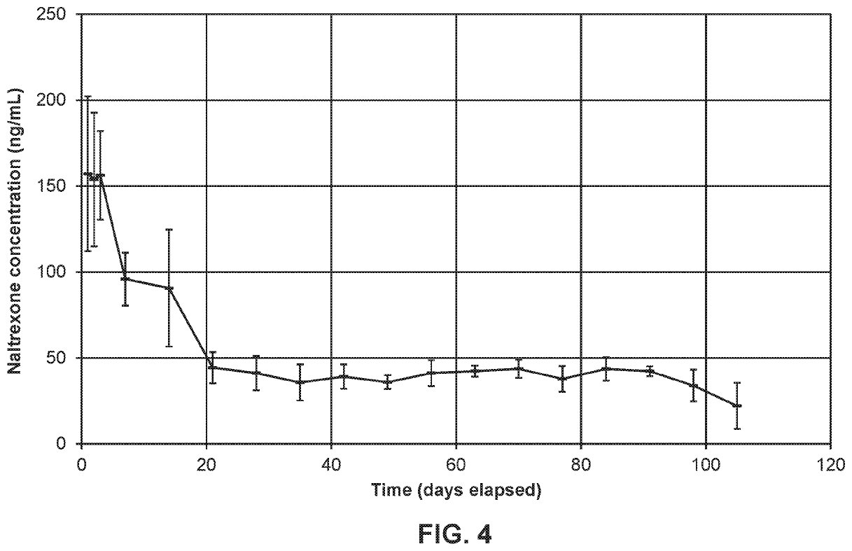

FIG. 4 shows the weight-normalized plasma levels of naltrexone, in ng/mL, as a function of time in days after implantation. Following an initial burst, naltrexone plasma levels reaches a steady, constant rate of release about 20 days after implantation, and provided an essentially constant rate of release for about 80 days. Accordingly, a device that comprises a reservoir comprising a formulation of an opioid antagonist in an amount sufficient to provide a therapeutic effect for a period of at least about 30 days, 60 days, 90 days, 100 days, 120 days 150 days, or 180 days and an organic acid that maintains a pH of the formulation when hydrated in its environment use of less than or equal to 11.5 for the period is provided. The formulation provides a release rate of the opioid antagonist sufficient to provide a therapeutic dose of the agent for the period.

Other drug delivery devices are known in the art and are suitable for delivery of the compositions described herein. That is, the compositions described herein are useful for a variety of devices, including those that comprise a drug reservoir for retaining the small molecule therapeutic agent and organic acid formulation and those that have a substrate or matrix that can hold or contain the formulation. Controlled drug release devices suitable for use in the present invention generally can provide for delivery of the drug from the device at a selected or otherwise patterned amount and/or rate to a selected site in the subject. The drug delivery device must be capable of containing an sufficient amount of the formulation to elute a therapeutically effective amount of the opioid antagonist for up to 12 month following implantation.

Accordingly, in another aspect, an implantable device is contemplated. The device comprises a reservoir comprising a formulation of an opioid antagonist, the formulation comprising (i) an amount of the therapeutic agent to provide substantially zero-order release of the therapeutic agent for a delivery period of at least about 30 days and up to 1 year at a rate that provides a therapeutic effect (opioid antagonism), and (ii) a molar ratio of organic acid to therapeutic agent ranging from 0:1 to 1:1 to 1:1, from 0.1:1 to 0.99:1, from 0.1:1 to 098:1, from 0.1:1 to 0.97:1, from 0.1:1 to 0, 96:1, from 0.1:1 to 0.95:1, or from 0.1:1 to 0.90:1. In other embodiments, the organic acid is a diprotic acid or a triprotic acid, the molar ratio of organic acid to therapeutic agent can range from 0.1:1 to 0.5:1, 0.1:1 to 0.45:1, 0.1:1 to 0.4:1, 0.1:1 to 0.35:1, 0.1:1 to 0.33:1, 0.1:1 to 0.3:1, 0.1:1 to 0.25:1, 0.1:1 to 0.2:1. 0.1:1 to 0.15:1. The formulation and device provide a method for sustained, controlled delivery of the opioid antagonist at a rate that achieves a therapeutically effective amount for the delivery period.

In one embodiment, the formulation comprising an opioid antagonist and organic acid at a molar ratio ranging from 0:1 to 1:1 is in a dry form. For example, the dry formulation may be present in the reservoir of a device as a powder, a tablet or a film. In one embodiment, the device when in use, in vitro or in vivo, imbibes fluid through a hydrophilic membrane from the surrounding environment to hydrate the dry formulation, thus forming in situ an aqueous suspension.

The drug delivery device can be implanted at any suitable implantation site using methods and devices well known in the art. As noted infra, an “implantation site” is a site within the body of a subject at which a drug delivery device is introduced and positioned, and can be considered an environment of use. Implantation sites include, but are not necessarily limited to, a subdermal, subcutaneous, intramuscular, or other suitable site within a subject's body. Subcutaneous implantation sites are preferred because of convenience in implantation and removal of the drug delivery device. Exemplary subcutaneous delivery sites include under the skin of the abdomen, arm, shoulder, neck, back, or leg. Sites within a body cavity are also suitable implantation sites. Methods for implanting or otherwise positioning drug delivery devices for subcutaneous delivery of a drug are well known in the art. In general, placement of the drug delivery device will be accomplished using methods and tools that are well known in the art, and performed under aseptic conditions with at least some local or general anesthesia administered to the subject.

### Methods of Treatment

In other aspects, methods of treatment using the compositions and devices described herein are contemplated. In one embodiment, a method for sustained, controlled delivery of an opioid antagonist is contemplated, where a composition or a delivery device comprising a composition as described herein is provided.

One or a plurality of devices are provided for administration to a human subject to treat opioid use disorder or alcohol abuse disorder. In one embodiment, between 1-4 devices are provided, and in another embodiment, the 1-4 devices are administered to the subject. One route of administration is via implantation, and a preferred implantation site is subcutaneous implantation, such as in the subcutaneous tissue in the abdomen or the arm. Implantation is accomplished, for example, with an implanter tool and other instruments provided in a surgical kit. Following implantation, the device(s) elutes sufficient drug in vivo to achieve a plasma concentration sufficient for therapy for a period of at least about 1 month and up to 3 months, 4 months, 6 months or 12 months. For example, when the opioid antagonist is naltrexone base, the target plasma concentration is >1-2 ng/mL for at least 1 month and up to 12 months, as this level of exposure is known to antagonize a typical dose of an ingested opioid. In other embodiment, the device comprises an amount of opioid antagonist to provide a therapeutic blood (plasma or serum or blood) concentration for between 1-12 months, 1-8 months, 1-6 months, 1-4 months, 1-3 months, 2-12 months, 2-10 months, 2-8 months, 2-6 months, 2-4 months, 3-12 months, 3-10 months, 3-9 months, 3-8 months, 3-6 months, or 3-4 months. Sufficient output of opioid antagonist from the device can be achieved, for example, by balancing the shape and size of the device, mass of drug loaded and the porosity and diffusive surface area of the porous partition and number of implanted devices.

In one embodiment, a device with an internal volume of between about 500-1000 μL comprises a composition of naltrexone base. The amount of naltrexone base in the device is sufficient to provide a therapeutically relevant plasma level of naltrexone and its active metabolite, 6-β-naltrexol, for a period of at least about 1 month, at least about 2 months, at least about 3 months, or for between 1-12 months, 1-6 months, 1-3 months, or 2-12 months, 2-6 months or 2-3 months.

In another embodiment, a method for maintaining therapeutic plasma levels of an opioid antagonist is contemplated, thus delaying relapse for stable, previously medicated patients for at least 4 weeks is contemplated.

Based on the foregoing, the compositions described herein comprising an organic acid and an opioid antagonist in a molar ratio ranging from 0:1 to 1:1 provide release of the opioid antagonist for an extended period of time—for at least about 14 days or for at least about 30 days and up to one year—at a constant rate that approaches zero-order release kinetics for the period. The composition comprises the therapeutic agent in an amount sufficient for a therapeutic dose of the agent for period, and an amount of the organic acid to maintain an elevated concentration of soluble therapeutic agent within the device throughout the delivery period, relative to that achievable by saturating the aqueous compartment with the free base form of the drug. The composition is, in some embodiments, retained in a drug delivery system (or device) and when placed in an environment of use (such as a subcutaneous implantation site, e.g., plasma or interstitial fluid with a constant pH˜7.4) produces a constant concentration gradient between the device interior and its environment of use that facilitates a constant release rate (near zero-order kinetics) of the therapeutic agent over time.

### III. Examples

The following examples are illustrative in nature and are in no way intended to be limiting.

### Example 1

### Formulation Comprising Naltrexone as a Small Molecule Therapeutic Agent and an Organic Acid

Naltrexone base was compounded with anisic acid at acid:drug ratios of 0:1 or 1.1 (molar basis), tableted with polyvinylpyrrolidone as a binder and stearic acid as a lubricant (12% and 2% of the final formulation mass, respectively), and loaded into delivery devices equipped with 0.1 micron polyvinylidene fluoride (DURAPORE®) membranes. In a subset of devices, approximately 75% of the available membrane surface area was blocked to measure the influence of surface area upon output rate. All devices were vacuum back-filled with phosphate buffer and transferred to jars containing a volume (˜100 mL) of the same buffer. The sealed jars were then incubated at 37° C., and small aliquots (˜500 μL) of receiving buffer were withdrawn at selected time points to quantify the released drug by high pressure liquid chromatography (HPLC). Release of naltrexone is shown in FIG. 2.

### Example 2

### Dry Formulation Comprising Naltrexone Base and an Organic Acid

Compressed pellets were prepared from mixtures of naltrexone base, an organic acid excipient, polyvinylpyrrolidone (12% by mass as a binder) and stearic acid (2% as a tablet press lubricant). The organic acid excipients were p-anisic acid or p-aminobenzoic acid, in the formulation at a mole ratio or 25%, 50%, or 100% relative to naltrexone (0.25:1, 0.5:1 and 1:1 organic acid:naltrexone mole ratios). Compressed tables of naltrexone base, PVP and stearic acid were prepared as a control.

Drug delivery devices with a 0.1 micron polyvinylidene fluoride (DURAPORE®) membranes and as depicted in FIG. 1 were filled with the compressed pellets (n=3). The in vitro release of naltrexone was measured following the process of Example 1. Results are shown in FIG. 3.

### Example 3

### Dry Formulation Comprising Naltrexone Base and an Organic Acid

Compressed pellets were prepared from a mixture of naltrexone base, p-anisic acid, polyvinylpyrrolidone (12% by mass as a binder) and stearic acid (2% as a tableting lubricant). The acid excipient was combined with naltrexone in a mole ratio of 1:1.

Prototype devices were prepared by loading cylindrical reservoirs with pellets (approximately 320 mg/device). These were then capped at each end with 0.1 micron polyvinylidene fluoride (DURAPORE®) membranes as depicted in FIG. 1. Prototype devices were terminally sterilized and implanted into male Sprague-Dawley rats (n=5). Plasma samples and animal weights were obtained at protocol-prescribed time points over a period of approximately 100 days, and plasma concentrations of naltrexone were obtained by liquid chromatography/mass spectroscopy. Results are shown in FIG. 4.

Devices recovered from experimental animals were subjected to mass balance analysis. Residual naltrexone was quantified and compared to the initial quantity loaded for each device. Based on this analysis, test devices eluted naltrexone at an average rate of approximately 2.5 mg/day.

1. A composition, comprising:
an aqueous mixture comprising an organic acid and an opioid antagonist at an organic acid:opioid antagonist mole ratio of less than or equal to 1:1, wherein the organic acid (i) has a water solubility at room temperature between 0.1 and 10 g/L, (ii) has a molar mass of less than 400 grams per mole, and/or (iii) maintains a pH of the mixture in its environment of use that is between about 3.0-11.5.

2. The composition of claim 1, wherein the opioid antagonist is naltrexone.

3. The composition of claim 1, wherein the organic acid anisic acid.

4. The composition of claim 1, wherein the opioid antagonist is nalmefene, nalorphine, nalodeine, levallorphan, xorphanol, oxilorphan, samidorphan, 6-beta-naltrexol, methylnaltrexone, diprenorphine or cyprodime.

5. (canceled)

6. The composition of claim 1, wherein the aqueous mixture comprises a buffer.

7. The composition of claim 6, wherein in the buffer is phosphate buffered saline.

8. The composition of claim 1, wherein the organic acid is an aromatic carboxylic acid.

9. (canceled)

10. The composition of claim 8, wherein the carboxylic acid is one having a carboxylic acid group bound to an unsubstituted benzene or pyridine ring.

11. The composition of claim 10, wherein the carboxylic acid is selected from the group consisting of benzoic acid, picolinic acid, nicotinic acid, and isonicotinic acid.

12. The composition of claim 8, wherein the carboxylic acid is one having a benzene ring and one electron-donating group with antioxidant properties.

13. The composition of claim 12, wherein the carboxylic acid is selected from the group consisting of o-anisic acid, m-anisic acid, p-anisic acid, p-aminobenzoic acid (PABA), o-aminobenzoic acid (anthranilic acid), o-toluic acid, m-toluic acid, p-toluic acid and salicylic acid.

14. The composition of claim 8, wherein the carboxylic acid is one having one benzene ring and two electron donating groups with antioxidant properties.

15. The composition of claim 1, wherein the amount of the opioid antagonist is sufficient to provide therapy for at least 30 days.

16. The composition of claim 1, wherein the composition is in a dry form.

17. An implantable device, comprising:
(a) a reservoir containing a formulation of an opioid antagonist, the formulation comprising (i) an amount of the opioid antagonist sufficient to provide the therapeutic effect for a period of at least about 30 days, (ii) an organic acid that maintains a pH of the formulation when hydrated in its environment use of less than or equal to 11.5 for the delivery period, and (iii) a release rate which provides a therapeutic dose of the agent for the period, and
(b) a porous partition in communication with the reservoir.

18. The device of claim 17, wherein the opioid antagonist is naltrexone.

19. The device of claim 17, wherein the opioid antagonist is nalmefene, nalorphine, nalodeine, levallorphan, xorphanol, oxilorphan, samidorphan, 6-beta-naltrexol, methylnaltrexone, diprenorphine or cyprodime

20. The device of claim 17, wherein the porous partition has a porosity of 60-75% and a surface area of about 2-22 mm².

21. The device of claim 17, wherein the reservoir has an outer diameter of about 2-6 mm and a length of about 30-80 mm.

22. The device of claim 17, wherein the formulation when hydrated has an aqueous phase with a pH value between about 3.0-11.5.

23. (canceled)

24. (canceled)

25. The device of claim 17, wherein the formulation is in dry form.

26. The device of claim 25, wherein the formulation is a powder, a tablet or a film.

27. The device of claim 25, wherein the dry formulation hydrates in the presence of an aqueous solution.

28. The device of claim 17, wherein the opioid antagonist is released from the device at a rate that provides a therapeutically effective amount for the period.

29. The device of claim 17, wherein the organic acid has a water solubility at room temperature of between about 0.1 and 10 g/L and a pKa between about 3 and 6.

30. A method for sustained, controlled delivery of a small molecule opioid antagonist, comprising:
providing a composition according to claim 1.

31. (canceled)

32. (canceled)

33. (canceled)

34. (canceled)
